(19) United States
(12) Patent Application Publication
(10) Pub. No.: US 2026/0083697 A1
(43) Pub. Date: Mar. 26, 2026

(54) COMPOSITION AND USE OF SAME FOR CONTROLLING INFECTIONS IN AQUATIC ORGANISMS

(71) Applicant: B. G. NEGEV TECHNOLOGIES AND APPLICATIONS LTD., AT BEN-GURION UNIVERSITY, Beer-Sheva (IL)

(72) Inventors: Inna KHOZIN-GOLDBERG, Beer Sheva (IL); Dina ZILBERG, Beer Sheva (IL); Boris ZORIN, Beer Sheva (IL); Ji Hyun KIM, Beer Sheva (IL); Arunjyothi JAWAJI, Beer Sheva (IL)

(73) Assignee: B. G. NEGEV TECHNOLOGIES AND APPLICATIONS LTD., AT BEN-GURION UNIVERSITY, Beer-Sheva (IL)

(21) Appl. No.: 19/108,800

(22) PCT Filed: Sep. 5, 2023
(86) PCT No.: PCT/IL2023/050950

§ 371 Date: Mar. 5, 2025

Related U.S. Application Data
(60) Provisional application No. 63/403,814, filed on Sep. 5, 2022.

(51) Int. Cl. A61K 31/232; A61K 31/201; A61K 31/202; A61P 33/02

(52) U.S. Cl. CPC A61K 31/232; A61K 31/201; A61K 31/202; A61P 33/02

(57) ABSTRACT

The present application, provides, inter alia, a composition including an esterified fatty acid, including a fatty acid comprising 4 to 24 carbon atoms, for use in a method for prevention or treatment of a monogenean infection in an aquatic organism in need thereof.

### CROSS-REFERENCE TO RELATED APPLICATIONS

This application claims the benefit of priory of U.S. Provisional Application No. 63/403,814, titled “COMPOSITION AND USE OF SAME FOR CONTROLLING INFECTIONS IN AQUATIC ORGANISMS”, filed 5 Sep. 2022, the contents of which are incorporated herein by reference in their entirety.

### FIELD OF INVENTION

The present invention is directed to, inter alia, composition and methods of using same, such as for controlling pathogenic infections affecting aquatic animals.

### BACKGROUND

Microalgae-based biotechnologies have become important tools in the focus of developing various commercially important products of commercial importance, including high-value natural biomolecules, such as carotenoids, exopolysaccharides, LC-PUFA, and others. Microalgae-based products are currently used in various sectors and deployed in different applications, such as the production of health-beneficial nutraceuticals, ingredients for human and animal nutrition, functional foods, prebiotics, biofuels, and cosmetics. A growing number of microalgae are used in the aquacultural industry as green water and feed ingredients. Microalgae represent a potent resource for aquacultural feed ingredients, due to their content of beneficial essential compounds, such as well-balanced proteins, LC-PUFA compositions, and immunomodulatory molecules. However, despite their numerous advantages, the production scales of microalgae-based technologies are still insufficient, and the production costs are high. In order to fully exploit microalgae's potential as renewable and sustainable natural resources, the biorefinery approach needs to be implemented. In this approach, various extraction and fractionation technologies are integrated for multipurposed biomass utilization. Once the target compounds are extracted from the microalgae, large amounts of residual biomass remain. The residue may contain a range of valuable compounds, such as carbohydrates, lipids, proteins, vitamins, and other molecules that potentially possess nutritional, health-promoting, and other beneficial properties that can be utilized.

There are several examples of microalgae and microalgae-derived fractions and preparations used as a treatment against microorganisms infection in fish. Organic solvent extracts of the green microalgae Chlorella pyrenoidosa and Scenedesmus quadricauda have demonstrated antifungal activity against the fungal pathogen Fusarium monofiliform, which causes black gill disease in shrimp. Tetraselmis suecica homogenates showed antibacterial activity against several Vibrio species, including important shrimp pathogens such as Vibrio alginolyticus, Vibrio anguillarum, Vibrio parahaemolyticus, and Vibrio vulnificus. The extract of the diatom Phaeodactylum tricornutum showed antibacterial properties against the Gram-positive Streptococcus iniae.

Guppies are one of the most important ornamental tropical, freshwater fish originating from the Caribbean and Central and South America. Guppies are natural hosts for monogenean parasites, which are commonly occurring ectoparasites of freshwater fish (Bakke et al. 2002). Gyrodactylus spp. are monogenean flatworms that reproduce directly on the skin, with a short life cycle. They are viviparous, thus they carry an embryo in their uterus; their lifecycle does not include an intermediate host, and so population growth is high. The parasites cause damage to the host fish by attaching to it with sclerotized hooks, feeding on its mucus and epithelial cells. Heavy gill infestations may cause severe hyperplasia of the gill filament epithelium, interfering with respiratory function, and may be a direct cause of death. The hook attachment to the fish skin causes tissue damage, resulting in increased mucous production, which disrupts the skin's protective functions and promotes secondary bacterial or fungal infection, often resulting in death. Parasite transmission occurs through direct contact between fish, as well as through the water.

Infection with monogenean parasites is not unique to guppies; different fish species are affected by a range of parasite species from the class Monogenea. These parasites are difficult to control, and various chemical treatments have been used against them. The conventional treatment for monogenean infections in fish is based on chemotherapy, including praziquantel, organophosphates, and formalin (which has very limited efficacy). Thus, these therapeutants pose associated problems of low efficacy, host toxicity, and environmental and human health concerns. The risk of human toxicity by treatment chemicals substantially increases when they are applied in closed production setups. Moreover, when applied repeatedly, chemical treatment leads to resistance development in the parasite. In recent years, the need has been recognized to move away from the traditionally used chemicals and to find alternative strategies of disease control and new natural treatments that are effective in promoting fish health and are also safe to use.

In a recent study, the use of the diatom P. tricornutum against G. turnbulli was examined and led to the development of an effective extract and identification of the active compounds, which were free fatty acids (FFAs). Phaeodactylum tricornutum is a fast-growing marine diatom, which is commercially produced by several biotech companies, for example, AlgaTech (Israel), Algahealth (Israel), ThinOgen (from macroalgae) as a source of nutraceuticals and/or food additives, mainly the keto-carotenoid fucoxanthin. Since fucoxanthin constitutes only a small percentage of the microalgal biomass, a large amount of algal by-product (or essentially waste biomass) is generated, which contains, among other ingredients, fatty acids, including the omega-3 LC-PUFA eicosapentaenoic acid (EPA), which has been found to be effective against G. turnbulli infection in guppies.

### SUMMARY

The present invention, in some embodiments, is based, at least in part, on the findings that a residual biomass from P. tricornutum fucoxanthin extraction was utilized to prepare an anti-parasitic treatment against the monogenean infection, aiming to develop a cost-effective natural treatment for the disease. Such a development would benefit both the aquacultural industry, by producing a natural treatment solution, and the algal producers, by generating another additional product from their cultured algae.

According to one aspect, there is provided a method for preventing or treating a pathogenic infection in an aquatic organism in need thereof, the method comprising administering to the aquatic organism a therapeutically effective amount of an esterified fatty acid, comprising a fatty acid chain of 4 to 24 carbon atoms.

According to another aspect, there is provided a composition comprising an esterified fatty acid, comprising a fatty acid chain of 4 to 24 carbon atoms, and an agriculturally or aquaculturally acceptable carrier, for prevention, treatment, or both, of a pathogenic infection in an aquatic organism in need thereof.

In some embodiments, the esterified fatty acid is ethyl ester, methyl ester, butyl ester, propyl ester, or a combination thereof.

In some embodiments, the esterified fatty acid comprises a plurality of types of esterified fatty acids, wherein each of the esterified fatty acids comprises a fatty acid chain of 4 to 24 carbon atoms.

In some embodiments, the esterified fatty acid comprises an esterified fatty acid being selected from the group consisting of: eicosapentaenoic acid (EPA), myristic acid, palmitic acid, palmitoleic acid, and any combination thereof.

In some embodiments, the esterified fatty acid comprises an esterified EPA.

In some embodiments, the fatty acid chain is of 9 to 22 carbon atoms.

In some embodiments, the fatty acid is a saturated fatty acid.

In some embodiments, the fatty acid is an unsaturated fatty acid, with 1 to 6 double bonds.

In some embodiments, the composition further comprises a detergent.

In some embodiments, the detergent is present in the composition in an amount of 0.1-5% by weight of the composition.

In some embodiments, the pathogenic infection excludes bacterial infection.

In some embodiments, the administering comprises contacting a body of water comprising the aquatic organism with the esterified fatty acid.

In some embodiments, contacting the body of water is with an amount of 0.1 to 500 μg of the esterified fatty acid per ml of water of the body of water.

In some embodiments, contacting the body of water is with an amount of 1 to 50 μg of the esterified fatty acid per ml of water of the body of water.

In some embodiments, the aquatic organism is selected from the group consisting of: fish, crustacean, mollusk, and any combination thereof.

In some embodiments, the pathogenic infection comprises any one of a monogenean infection, a protozoan infection, or both.

In some embodiments, the monogenean infection is induced by Gyrodactylus turnbulli.

In some embodiments, the protozoan infection is induced by a protozoan belonging to any one of the genera Trichodina and Tetrahymena.

In some embodiments, the detergent comprises Kolliphor EL.

In some embodiments, the composition comprises an extract of a microalga, or any fraction thereof.

In some embodiments, the microalga is selected from the group consisting of: Phaeodactylum tricornutum, Odontella aurita, Nitzschia laevis, Nannochloropsis oceanica, Nannochloropsis limnetica, Nannochloropsis oculata, Microchloropsis gaditana, Microchloropsis salina, Monodopsis subterranean, Tribonema sp, and any combination thereof.

In some embodiments, the composition is administered or supplemented into a body of water comprising the aquatic organism, such that a concentration of 1 to 50 μg of the esterified fatty acid per ml of water of the body of water is obtained.

In some embodiments, the composition is formulated for administration to a body of water.

In some embodiments, the body of water comprises the aquatic organism.

In some embodiments, the composition is formulated for administration to an aquatic organism while residing, being cultured, or both, in the body of water.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 includes a scheme of a non-limiting workflow for developing the extract and preparations from P. tricornutum residue against G. turnbulli, and analysis to determine the active compounds. Antiparasitic activity was tested in situ and in vivo; active compounds were analyzed by thin-layer chromatography (TLC) and gas chromatography coupled with a flame ionization detector (GC-FID). Fractions obtained from TLC were tested in situ to identify the active fractions.

FIGS. 2A-2C include graphs showing antiparasitic effect of residue extract and preparations (FFA & FAEE) with and without the addition of the food-grade detergent Kolliphor (K) and sonication(S) prior to analysis. Parasites were observed for mortality over time for 240 min. Gyrodactylus turnbulli was exposed to the following preparations: (2A) residue extract (RE) and RE with Kolliphor and sonication (RE-K-S); (2B) FFA and FFA with Kolliphor and sonication (FFA-K-S); and (2C) FAEE with Kolliphor and sonication (FAEE-K-S). The numbers represent the tested concentrations of the solution applied at μL mL⁻¹. n=3 wells with at least five observed parasites per well.

FIGS. 3A-3C include box plots showing in vivo antiparasitic effects of residue extract and preparations with added 1% Kolliphor and sonication. Guppies infected with Gyrodactylus turnbulli were bath treated with the residue extract and preparations (1.25 and 2.5 μL mL⁻¹) for 24 h, and Ethanol (sp) K-S served as a control (2.5 μL mL⁻¹). (3A) Bath treatment with RE (RE-K-S). (3B) Bath treatment with FFA (FFA-K-S). (3C) Bath treatment with FAEE (FAEE-K-S). The number of parasites were counted on the tail fin of each individual fish before and after each treatment application (n=10 fish per treatment group). * Significant difference in the number of parasites before and after treatment within treatment groups; different lowercase letters denote significant differences between treatment groups (p<0.001). The values above the boxes denote infection prevalence (i.e., % of infected fish).

FIG. 4 includes a micrograph showing separation by one dimension thin-layer chromatography (1D-TLC) of P. tricornutum residue extract (RE), free fatty acids (FFAs) produced from the RE, and fatty acid ethyl esters (FAEEs) produced from the original residue powder. Lipid spots were visualized by exposing the plate to iodine vapors. Lipid standards are shown on the left.

FIG. 5 includes a vertical bar graph showing dose-dependent antiparasitic effect of fatty acids in their ethyl ester form as tested in situ. Selected were the four FAs that appeared at the highest level in the residue and its extracts and preparations, as pure commercial compounds: myristic acid (14:0), palmitic acid (16:0), palmitoleic acid (16:1n7), and EPA (20:5n3). The analysis was carried out with the addition of Kolliphor (K) and sonication (S). Time to reach 50% parasite mortality was recorded.

FIG. 6 includes a graph showing a toxicity test of control treatments. Gyrodactylus turnbulli was exposed to the various solutions containing a solvent and a detergent, which were used for the preparation of the algal extract and preparations (FFA and FAEE), to reveal their non-toxic levels: Ethanol; Ethanol-K (ethanol with addition of the food-grade detergent Kolliphor EL, at 1% in the stock solution); Ethanol-K(sp)* (Ethanol-K that underwent a saponification procedure); Ethanol (sp)-K** (Ethanol that underwent a saponification procedure, and the addition of Kolliphor EL prior to analyses); and Ethanol-K1-S(Ethanol-K with added sonication, S). The numbers represent the tested concentrations at μL ml⁻¹. n=3 wells with at least five parasites per well, which were observed for each treatment. *Ethanol-K (sp): a procedure applied for FFA preparation. **Ethanol (sp)-K: a procedure applied for FFA preparation.

FIG. 7 include a micrograph showing histopathological analysis of gills from fish following a 24-h bath treatment in FAAEs from P. tricornutum residue powder, at a concentration of 2.5 μL mL⁻¹. No adverse effect was evident on the fish's gills.

FIG. 8 includes a graph showing anti-parasitic effect of fatty acid ethyl esters (FAEE), prepared by direct transethylation applied to the P. tricornutum residue powder. FAEE were dissolved in ethanol, Kolliphor (1% in the stock solution) and sonication(S) were applied prior to analysis (FAEE-K-S). Trichodina was exposed to fatty acid ethyl esters (FAEE (R)-K) and observed for mortality over time for 240 mins. For control, water was used. The numbers represent the tested concentrations at μl/mL. n=3 wells with at least 50 parasites per well were observed.

FIG. 9 includes a graph showing in vivo antiparasitic effect against Trichodina in Barramundi by FAEE from algal P. tricornutum residue. Detergent Kolliphor 1% (K) and sonication(S) were added to FAEE prior to analysis: FAEE-K-S; and FAEE-K-S. Barramundi infected with Trichodina were bath-treated with the FAEE at 1.25 and 2.5 μL mL⁻¹. Ethanol-K-S was served as a control (2.5 μL mL⁻¹). The number of parasites were counted on the skin and gills (G) of each individual fish before and after treatment application (n=8 fish per treatment group) and parasites on gills were checked 24 h after treatment. *Significant difference in the number of parasites before and after treatment within treatment groups; different lowercase letters denote significant differences between treatment groups (p≤0.001).

FIG. 10 includes a graph showing in situ antiparasitic effect against Sea lice by FAEEs of whole Biomass and Residue from the microalga P. tricornutum (a diatom). Sea lice were exposed to FAEE.B-K-S and FAEE.R-K-S extract at 20, 50 and 100 μl/mL with addition of 1% Kolliphor and Sonication prior to analysis. For the 100 μl/mL, the parasites were moved to clean water after 10 min of exposure (red arrow) after which recovery was evident.

FIG. 11A-11B include graphs showing in-situ antiparasitic effect against Gyrodactylus by commercial FAEEs. Gyrodactylus was exposed by commercial FAEEs at 18.75, 37.5, and 75 μM along with addition of 1% Kolliphor (K) and sonication(S). Parasites were observed for 50% (11A) and 100% (11B) mortality over time for 240 min. n=3 wells with at least 15 observed parasites per well.

FIGS. 12A-12B include graphs showing in-situ antiparasitic effect against Trichodina by commercial FAEEs. Trichodina was exposed by commercial FAEEs at 18.75, 37.5, and 75 μM along with addition of 1% Kolliphor (K) and sonication(S). Parasites were observed for 50% (12A) and 100% (12B) mortality over time for 240 min. n=3 wells with at least 15 observed parasites per well.

### DETAILED DESCRIPTION

According to some embodiments, there is provided a method for preventing or treating a pathogenic infection in an aquatic organism in need thereof.

In some embodiments, a pathogenic infection excludes a bacterial infection.

In some embodiments, the pathogenic infection comprises a monogenean infection, a protozoan infection, or both.

In some embodiments, a monogenean infection is induced by Gyrodactylus turnbulli.

In some embodiments, a protozoan infection comprises an infection induced by a protozoan belonging to the genera Trichodina.

In some embodiments, the method comprising administering to an aquatic organism a therapeutically effective amount of an esterified fatty acid. In some embodiments, administering comprises directly administering to an aquatic organism, such as by contacting or the like. In some embodiments, administering comprises indirectly administering, such as by supplementing into a body of water wherein the aquatic organism is cultured.

In some embodiments, a fatty acid, such as comprised by or included in an esterified fatty acid as disclosed herein, comprises a fatty acid chain of: at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24 carbon atoms, or any value and range therebetween. Each possibility represents a separate embodiment of the invention.

In some embodiments, a fatty acid chain, such as comprised by or included in an esterified fatty acid as disclosed herein, is of: 4-24 carbon atoms, 8-24 carbon atoms, 9-24 carbon atoms, 10-24 carbon atoms, 12-24 carbon atoms, 14-24 carbon atoms, 20-24 carbon atoms, or 22-24 carbon atoms. Each possibility represents a separate embodiment of the invention.

In some embodiments, an esterified fatty acid is ethyl ester, methyl ester, butyl ester, propyl ester, any salt thereof, or any combination thereof.

In some embodiments, an esterified fatty acid comprises a plurality of esterified fatty acids, as disclosed herein.

In some embodiments, an esterified fatty acid comprises a fatty acid derived or obtained from a natural source. In some embodiments, an esterified fatty acid comprises a fatty acid derived or obtained from an animal. In some embodiments, an esterified fatty acid comprises a fatty acid derived or obtained from a plant. In some embodiments, an esterified fatty acid comprises a fatty acid derived or obtained from an arthropod. In some embodiments, an esterified fatty acid comprises a fatty acid derived or obtained from an insect. In some embodiments, an esterified fatty acid comprises a fatty acid derived or obtained from a fly. In some embodiments, an esterified fatty acid comprises a fatty acid derived or obtained from a black Solider fly. In some embodiments, an esterified fatty acid comprises a fatty acid derived or obtained from a larva of an arthropod, insect, fly, or any combination thereof. In some embodiments, an esterified fatty acid comprises a fatty acid derived or obtained from a natural source comprising and/or enriched with a 12:0 fatty acid.

In some embodiments, an esterified fatty acid comprises a plurality of types esterified fatty acids.

In some embodiments, each of the esterified fatty acids comprises a fatty acid chain of 4 to 24 carbon atoms.

In some embodiments, each of the esterified fatty acids of the plurality of types of esterified fatty acids comprises a fatty acid chain of 4 to 24 carbon atoms.

In some embodiments, an esterified fatty acid comprises an esterified fatty acid being selected from: eicosapentaenoic acid (EPA), myristic acid, palmitic acid, palmitoleic acid, a salt thereof, or any combination thereof.

In some embodiments, the esterified fatty acid comprises an esterified EPA.

In some embodiments, the fatty acid comprises a fatty acid chain of 9 to 22 carbon atoms.

As used herein, the terms “ethyl butyrate”, “4:0”, “short chain fatty acid of 4 carbons” are interchangeable. As used herein, the terms “ethyl hexanoate”, “6:0”, “short chain fatty acid of 6 carbons” are interchangeable. As used herein, the terms “ethyl octanoate”, “8:0”, “short chain fatty acid of 8 carbons” are interchangeable. As used herein, the terms “ethyl nonanoate”, “9:0”, “short chain fatty acid of 9 carbons” are interchangeable. As used herein, the terms “ethyl decanoate”, “10:0”, “short chain fatty acid of 10 carbons” are interchangeable. As used herein, the terms “ethyl laurate”, “12:0”, “saturated fatty acid of 12 carbons” are interchangeable. As used herein, the terms “ethyl myristate”, “14:0”, “saturated fatty acid of 14 carbons” are interchangeable. As used herein, the terms “ethyl palmitate”, “16:0”, “short chain fatty acid of 4 carbons” are interchangeable. As used herein, the terms “ethyl palmitoleate”, “16:1”, “monounsaturated fatty acid or MUFA of 4 carbons” are interchangeable. As used herein, the terms “ethyl ricinoleate”, “18:1-OH”, “monounsaturated fatty acid or MUFA of 18 carbons” are interchangeable. As used herein, the terms “ethyl linoleate” and “18:2n6” are interchangeable. As used herein, the terms “ethyl alpha linolenate” and “18:3n3” are interchangeable. As used herein, the terms “ethyl gamma linolenate” and “18:3n6” are interchangeable. As used herein, the terms “eicosapentaenoic acid”, “EPA”, and “20:5n3” are interchangeable.

In some embodiments, the fatty acid comprises a short chain fatty acid. In some embodiments, a short chain fatty acid comprises a fatty acid of 4 to 10 carbon atoms. In some embodiments, a short chain fatty acid comprises a fatty acid selected from: 4:0, 6:0, 8:0, 9:0, 10:0, or any combination thereof. In some embodiments, a short chain fatty acid comprises a fatty acid selected from: 6:0, 8:0, 9:0, 10:0, or any combination thereof.

In some embodiments, the pathogenic infection is induced by a Gyrodactylus parasite, and the short chain fatty acid used according to the method of the invention comprises a fatty acid selected from: 4:0, 6:0, 8:0, 9:0, 10:0, or any combination thereof. In some embodiments, the pathogenic infection is induced by a Gyrodactylus parasite, and the short chain fatty acid comprises a fatty acid selected from 9:0 or 10:0.

In some embodiments, the pathogenic infection is induced by a Trichodina parasite, and the short chain fatty acid used according to the method of the invention comprises a fatty acid selected from 9:0 or 10:0.

In some embodiments, the fatty acid comprises saturated fatty acid. In some embodiments, a saturated fatty acid comprises a fatty acid of 12 to 18 carbon atoms. In some embodiments, a saturated fatty acid comprises a fatty acid selected from: 12:0, 14:0, 16:0, 18:0, or any combination thereof. In some embodiments, a saturated chain fatty acid comprises a fatty acid selected from: 12:0, 14:0, 16:0, or any combination thereof.

In some embodiments, the pathogenic infection is induced by a Gyrodactylus parasite, and the saturated fatty acid used according to the method of the invention comprises or consists of the fatty acid 12:0.

In some embodiments, the pathogenic infection is induced by a Trichodina parasite, and the saturated fatty acid used according to the method of the invention comprises a fatty acid selected from 12:0, 14:0, 16:0, or any combination thereof.

In some embodiments, the fatty acid comprises unsaturated fatty acid. In some embodiments, an unsaturated fatty acid comprises a monounsaturated fatty acid (MUFA). In some embodiments, an unsaturated fatty acid comprises polyunsaturated fatty acid (PUFA), long chain polyunsaturated fatty acid (LC-PUFA), or both.

In some embodiments, a monounsaturated fatty acid or MUFA comprises or consists of the fatty acid 18:1-OH. In some embodiments, the pathogenic infection is induced by a Gyrodactylus parasite, and the MUFA used according to the method of the invention comprises or consists of the fatty acid 18:1-OH. In some embodiments, the pathogenic infection is induced by a Trichodina parasite, and the MUFA used according to the method of the invention comprises or consists of the fatty acid 18:1-OH.

In some embodiments, a polyunsaturated fatty acid (PUFA), long chain polyunsaturated fatty acid (LC-PUFA), or both, comprises a PUFA, LC-PUFA, or both, selected from: 18:2n6, 18:3n3, 18:3n6, 20:3n6, 20:5n3, or any combination thereof. In some embodiments, a polyunsaturated fatty acid (PUFA), long chain polyunsaturated fatty acid (LC-PUFA), or both, comprises a PUFA, LC-PUFA, or both, selected from: 18:3n3, 20:3n6, 20:5n3, or any combination thereof. In some embodiments, a polyunsaturated fatty acid (PUFA), long chain polyunsaturated fatty acid (LC-PUFA), or both, comprises a PUFA, LC-PUFA, or both, comprising or consisting of 20:5n3.

In some embodiments, the pathogenic infection is induced by a Gyrodactylus parasite, and the PUFA or LC-PUFA comprises a fatty acid used according to the method of the invention is selected from: 18:2n6, 18:3n3, 18:3n6, 20:5n3, or any combination thereof. In some embodiments, the pathogenic infection is induced by a Gyrodactylus parasite, and the PUFA or LC-PUFA used according to the method of the invention comprises or consists of the fatty acid 20:5n3.

In some embodiments, the pathogenic infection is induced by a Trichodina parasite, and the PUFA or LC-PUFA comprises a fatty acid used according to the method of the invention is selected from: 18:3n3, 20:3n6, 20:5n3, or any combination thereof.

In some embodiments, the unsaturated fatty acid comprises, includes, or is with 1 to 2, 1 to 3, 1 to 4, 1 to 5, and 1 to 6 double bonds.

In some embodiments, administering comprises contacting a body of water comprising an aquatic organism with an esterified fatty acid as disclosed herein.

In some embodiments, the esterified fatty acid is in a composition. In some embodiments, there is provided a composition comprising the esterified fatty acid, and an acceptable carrier. In some embodiments, the composition comprises the esterified fatty acid and an acceptable carrier. In some embodiments, the carrier is an agriculturally acceptable carrier, a pharmaceutically acceptable carrier, or both. In some embodiments, an agriculturally acceptable carrier comprises an aquaculturally acceptable carrier. In some embodiments, the composition is an agricultural composition. In some embodiments, the composition is an aquacultural composition.

In some embodiments, the esterified fatty acid is present in the composition in a concentration ranging from 1 and 300 μM, 1 and 200 μM, 1 and 100 μM, 1 and 80 μM, 1 and 50 μM, 1 and 30 μM, 1 and 15 μM, 1 and 10 μM, 5 and 300 μM, 5 and 150 μM, 5 and 75 μM, 5 and 50 μM, 10 and 300 μM, 10 and 150 μM, 10 and 75 μM, 10 and 50 μM, 15 and 150 μM, 15 and 75 μM, or 15 and 40 μM. Each possibility represents a separate embodiment of the invention.

In some embodiments, the composition comprises an esterified short chain fatty acid selected from: 9:0, 10:0, or both, in a concentration ranging between 10 and 100 μM, 15 and 80 μM, or 30 and 75 μM. Each possibility represents a separated embodiment of the invention. In some embodiments, the method comprises treating a pathogenic infection induced by Gyrodactylus, Trichodina, or both, in an aquatic organism in need thereof by administering to the aquatic organism a therapeutically effective amount of a composition comprising an esterified short chain fatty acid selected from: 9:0, 10:0, or both, in a concentration ranging between 10 and 100 μM, 15 and 80 μM, or 30 and 75 μM. Each possibility represents a separated embodiment of the invention.

In some embodiments, the composition comprises an esterified saturated fatty acid selected from: 12:0, 14:0, 16:0, or any combination thereof, in a concentration ranging between 20 and 100 μM, 25 and 80 μM, or 30 and 75 μM. Each possibility represents a separated embodiment of the invention. In some embodiments, the method comprises treating a pathogenic infection induced by Trichodina in an aquatic organism in need thereof by administering to the aquatic organism a therapeutically effective amount of a composition comprising an esterified saturated fatty acid selected from: 12:0, 14:0, 16:0, or any combination thereof, in a concentration ranging between 20 and 100 μM, 25 and 80 μM, or 30 and 75 μM. Each possibility represents a separated embodiment of the invention.

In some embodiments, the composition comprises an esterified MUFA comprising or consisting of 18:1-OH in a concentration ranging between 10 and 100 μM, 15 and 80 μM, or 30 and 75 μM. Each possibility represents a separated embodiment of the invention. In some embodiments, the method comprises treating a pathogenic infection induced by Trichodina in an aquatic organism in need thereof by administering to the aquatic organism a therapeutically effective amount of a composition an esterified MUFA comprising or consisting of 18:1-OH in a concentration ranging between 10 and 100 μM, 15 and 80 μM, or 30 and 75 μM. Each possibility represents a separated embodiment of the invention. In some embodiments, the method comprises treating a pathogenic infection induced by Gyrodactylus in an aquatic organism in need thereof by administering to the aquatic organism a therapeutically effective amount of a composition an esterified MUFA comprising or consisting of 18:1-OH in a concentration ranging between 20 and 100 μM, 25 and 80 μM, or 30 and 75 μM. Each possibility represents a separated embodiment of the invention.

In some embodiments, the composition comprises an esterified PUFA, LC-PUFA, or both, selected from: 18:2n6, 18:3n3, 18:3n6, 20:3n6, 20:5n3, or any combination thereof, in a concentration ranging between 20 and 100 μM, 25 and 80 μM, or 30 and 75 μM. Each possibility represents a separated embodiment of the invention. In some embodiments, the method comprises treating a pathogenic infection induced by Trichodina in an aquatic organism in need thereof by administering to the aquatic organism a therapeutically effective amount of a composition comprising an esterified PUFA, LC-PUFA, or both, selected from: 18:3n3, 20:3n6, 20:5n3, or any combination thereof, in a concentration ranging between 20 and 100 μM, 25 and 80 μM, or 30 and 75 μM. Each possibility represents a separated embodiment of the invention. In some embodiments, the method comprises treating a pathogenic infection induced by Gyrodactylus in an aquatic organism in need thereof by administering to the aquatic organism a therapeutically effective amount of a composition comprising an esterified PUFA, LC-PUFA, or both, selected from: 18:2n6, 18:3n3, 18:3n6, 20:5n3, or any combination thereof, in a concentration ranging between 20 and 100 μM, 25 and 80 μM, or 30 and 75 μM. Each possibility represents a separated embodiment of the invention. In some embodiments, the method comprises treating a pathogenic infection induced by Gyrodactylus in an aquatic organism in need thereof by administering to the aquatic organism a therapeutically effective amount of a composition comprising an esterified 20:5n3 fatty acid in a concentration ranging between 20 and 100 μM, 25 and 80 μM, or 30 and 75 μM. Each possibility represents a separated embodiment of the invention.

In some embodiments, the method comprises contacting a body of water with an amount of 0.1 to 500 μg of an esterified fatty acid per ml of water of the body of water.

In some embodiments, the method comprises contacting a body of water with an amount of 1 to 50 μg of an esterified fatty acid per ml of water of the body of water.

In some embodiments, an aquatic organism is selected from: fish, crustacean, mollusk, or any combination thereof.

In one embodiment, a fish belongs to the family of Salmonidae. In one embodiment, a fish is or comprises a salmon fish. In some embodiments, a fish belongs to the family of Latidae. In some embodiments, a fish belongs to the genus Lates. In some embodiments, a fish belonging to the family Latidae, and/or the genus Lates comprises or is Lates calcarifer.

According to some embodiments, there is provided a composition comprising an esterified fatty acid, comprising a fatty acid chain of 4 to 24 carbon atoms, and an agriculturally or aquaculturally acceptable carrier.

In some embodiments, the composition further comprises a detergent.

In some embodiments, the detergent comprises or consists of Kolliphor.

In some embodiments, the composition comprises an extract of a microalga, or any fraction thereof.

In some embodiments, the microalga is selected from: Phaeodactylum tricornutum, Odontella aurita, Nitzschia laevis, Nannochloropsis oceanica, Nannochloropsis limnetica, Nannochloropsis oculata, Microchloropsis gaditana, Microchloropsis salina, Monodopsis subterranean, Tribonema sp, or any combination thereof.

In some embodiments, the microalga comprises any microalgal species having a fatty acid profile equivalent or essentially similar to a microalga being selected from Phaeodactylum tricornutum, Odontella aurita, Nitzschia laevis, Nannochloropsis oceanica, Nannochloropsis limnetica, Nannochloropsis oculata, Microchloropsis gaditana, Microchloropsis salina, Monodopsis subterranean, Tribonema sp, or any combination thereof.

In some embodiments, the composition consists essentially of an esterified fatty acid, comprising a fatty acid chain of 4 to 24 carbon atoms, as disclosed herein.

As used herein, the phrases “consist essentially of” or “consisting essentially of” denote that a given compound or substance, e.g., an esterified fatty acid, comprising a fatty acid chain of 4 to 24 carbon atoms, as disclosed herein, constitutes the vast majority of the active ingredient's portion or fraction of the composition, e.g., an agricultural and/or aquacultural composition.

In some embodiments, consisting essentially of means that the esterified fatty acid, comprising a fatty acid chain of 4 to 24 carbon atoms, as disclosed herein constitutes at least 95%, at least 98%, at least 99%, or at least 99.9% by weight, of the active ingredient(s) of the agricultural and/or aquacultural composition, or any value and range therebetween. Each possibility represents a separate embodiment of the invention.

In some embodiments, the composition is being used for prevention, treatment, or both, of an infection in an aquatic organism in need thereof. In some embodiments, the infection is a monogenean infection. In some embodiments, the infection is an oligohymenophorean infection.

In some embodiments, the composition is being administered or supplemented into a body of water comprising an aquatic organism.

In some embodiments, the composition is administered or supplemented in a dose such that a concentration of 0.1 to 500 μg of an esterified fatty acid per ml of water of the body of water is obtained.

In some embodiments, the composition is administered or supplemented in a dose such that a concentration of 1 to 50 μg of an esterified fatty acid per ml of water of the body of water is obtained.

In some embodiments, the composition is administered or supplemented in a dose such that a concentration of 3 to 30 μg of an esterified fatty acid per ml of water of the body of water is obtained.

In some embodiments, the composition is formulated for agricultural or aquacultural applications.

In some embodiments, the composition is formulated for administration or supplementation into a body of water.

In some embodiments, the body of water comprises the aquatic organisms. In some embodiments, the aquatic organism is cultured or grown in the body of water. In some embodiments, the aquatic organism is an aquacultured organism.

According to some embodiments, the treating comprises ameliorating pathogenic infection or at least one symptom associated therewith in the aquatic organism.

As used herein, the terms “treatment” or “treating” of an infection, disease, disorder, or condition, encompasses alleviation of at least one symptom thereof, a reduction in the severity thereof, or inhibition of the progression thereof. Treatment need not mean that the infection, disease, disorder, or condition is totally cured. To be an effective treatment, a useful composition herein needs only to reduce the severity of an infection, disease, disorder, or condition, reduce the severity of symptoms associated therewith, or provide improvement to a subject's quality of life.

### General

As used herein, the term “about” when combined with a value refers to plus and minus 10% of the reference value. For example, a length of about 1,000 nanometers (nm) refers to a length of 1,000 nm±100 nm.

It is noted that as used herein and in the appended claims, the singular forms “a”, “an”, and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely”, “only”, and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Various embodiments and aspects of the present invention as delineated herein above and as claimed in the claims section below find experimental support in the following examples. Reference is now made to the following examples, which together with the above descriptions illustrate some embodiments of the invention in a non-limiting fashion.

Generally, the nomenclature used herein, and the laboratory procedures utilized in the present invention include chemical, molecular, biochemical, and cell biology techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); “Cell Biology: A Laboratory Handbook”, Volumes I-III Cellis, J. E., ed. (1994); The Organic Chemistry of Biological Pathways by John McMurry and Tadhg Begley (Roberts and Company, 2005); Organic Chemistry of Enzyme-Catalyzed Reactions by Richard Silverman (Academic Press, 2002); Organic Chemistry (6th Edition) by Leroy “Skip” G Wade; Organic Chemistry by T. W. Graham Solomons and, Craig Fryhle.

### Materials and Methods

### Fish and Parasite Lab Maintenance

Guppies were obtained from a commercial ornamental fish farm (Arava, Israel). Upon arrival, random fish were anesthetized and examined for infection with Gyrodactylus turnbulli by stereomicroscope (Zeiss Stemi 2000-C, Carl Zeiss AG, Germany). G. turnbulli identification was done based on Paladini et al., 2009. Fish were placed in 100-L tanks and subsequently moved to 30-L aquaria, where the infection was maintained. Tanks and aquaria were filled with de-chlorinated water and supplied with submerged biological filters and aeration. The water temperature was maintained at 26±2° C. Tanks and aquaria were siphoned every other day, and about 10% of the water was replaced. Water quality parameters were analyzed weekly using AquaMerck kits (Merck, Germany) and maintained at: ammonia: 0 mg L⁻¹, nitrite: 0.025 mg L⁻¹ and nitrate: 50 mg L⁻¹. Fish were fed once a day at about 2% of their body weight with commercial guppy feed (Mem Ornamental, BernAqua, Belgium). Experimental protocols were carried out in accordance with the principles of biomedical research involving animals from the Ben-Gurion University Committee for the Ethical Care and Use of Animals, authorization number; IL-79-10-2012.

Phaeodactylum tricornutum Residue

Phaeodactylum tricornutum residue was obtained from the microalgae-manufacturing company Algatech-Solabia (Kibbutz Ketura, Israel), which comprises the powdered residue of the algae after fucoxanthin extraction. This residue was stored at −80° C. until use.

Phaeodactylum tricornutum Residue Extract Preparation

Phaeodactylum tricornutum residue was extracted by a method previously developed (Kim et al., 2021). Briefly, 100 mg of dry residue powder was mixed with 2 mL of absolute ethanol and 20 mg of Kolliphor EL (1%, w/v; a food-grade nonionic emulsifier) in a glass tube and incubated for 4 h at room temperature under continuous mixing. The mixture was then centrifuged (2,558×g; 10 min and 21,380×g; 5 min; at 25° C.), and the supernatant was collected. The extract was aliquoted into small portions in Eppendorf tubes, filled with argon gas to prevent oxidation; tubes were sealed with parafilm, and then stored at −80° C. until analysis. Sonication for 10-30 sec in an ultrasonic bath (Transonic 460, Elma, Germany) was applied where specified before using the extract for analysis.

Free Fatty Acid (FFA) Preparation from P. tricornutum Residue Extract

The free fatty acid fraction was produced by basic hydrolysis applied to the algal residue extract. In the first step, saponification was performed by adding 40% of potassium hydroxide at 1:1 (v/v) proportion. The glass tube was filled with argon gas, sealed with a Teflon cap, and heated to 60° C. for 1 h in a sand bath under an argon atmosphere. The monophasic solution was then cooled and partitioned by adding hexane and separated into two phases by centrifugation (2,558×g; 5 min). The upper hexane phase, containing the non-saponified lipids, was discarded. The lower phase (hydroalcoholic phase), containing the saponifiable lipids and the potassium salts of fatty acids, was collected. The pH was adjusted to 3.0 with HCL; next, hexane (500 μL) was added to the liberated FFAs and then separated by centrifugation (2,558×g; 5 min). The same procedure was repeated twice, and all the hexane layers were combined and evaporated to dryness under nitrogen gas flow. The evaporated hexane fraction was weighed and dissolved in ethanol to the initial volume of the residue extract.

Fatty Acids Ethyl-Ester (FAEE) Preparation from P. tricornutum Residue by Direct Transmethylation

Algal residue powder was placed in Pyrex glass vials. Transmethylation was performed by adding 2% sulfuric acid (H₂SO₄) in anhydrous ethanol (v/v) under an argon atmosphere with continuous stirring. Sealed glass vials were placed in sand bath at 80° C. for 1.5 h. The transmethylation reaction was terminated by the addition of 1 mL of H₂O. The obtained fatty acid ethyl esters (FAEEs) were extracted with hexane; phase separation was achieved by centrifugation (2,558×g; 5 min). The same procedure was repeated twice, and all the hexane fractions were combined and evaporated to dryness under nitrogen gas flow. The evaporated hexane fraction was dissolved in ethanol to normalize to the amount of residue powder used for residue extraction.

Antiparasitic Effect of P. tricornutum Residue Against G. turnbulli

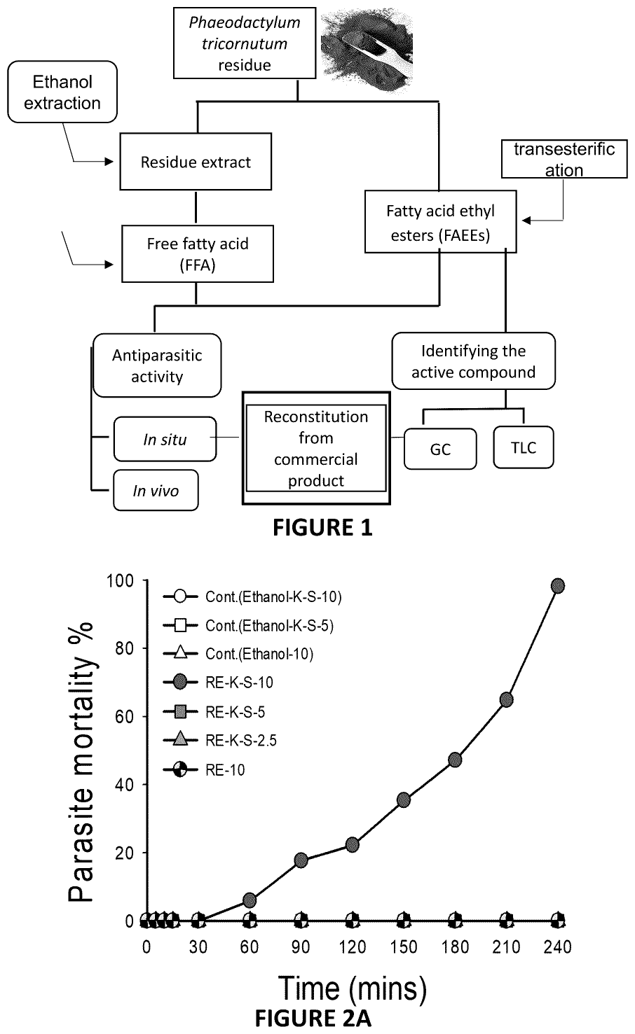

The overall research workplan for examining the antiparasitic effect of P. tricornutum residue extracts against G. turnbulli and identifying the active compounds responsible for this activity is presented in FIG. 1.

### In Situ Antiparasitic Effect

An in-situ assay was carried out, as described previously (Kim et al., 2021). Briefly, infected guppies (as confirmed by observation under a stereomicroscope (Richards and Chubb 1996)) were anesthetized with 250 ppm of Sedanol (Stockton-Aquamor Ltd., Israel) (Zorin et al., 2019) followed by euthanasia (brain pithing). Tail fin clips with more than four parasites were excised and transferred, using watchmaker's forceps, to a single well of a 24-well plate containing standardized water (STW: dechlorinated tap water with Na₂S₂O₃ at 0.37 mg L⁻¹ followed by autoclaving). Ethanol and STW, as well as the relevant solvents and preparation protocol used for extraction, were used as negative controls in each trial. Parasites were observed under an inverted microscope (Axiovert 40 CFL, Carl Zeiss AG) for 4 h, at intervals ranging from 5 to 30 min, during which detached and dead parasites were recorded. A total of 12-15 parasites (with three replicates) were analyzed for each treatment concentration.

Residue extract and preparations were tested at the following concentrations: residue extract at 2.5, 5 and 10 μL mL⁻¹, and FFA and FAEE at 2.5 and 5 μL mL⁻¹ (in ethanol). Selected pure active compounds were analyzed, based on the analysis of the FAEE composition. These included the ethyl esters of the following fatty acids: myristic acid (14:0), palmitic acid (16:0), palmitoleic acid (16:1n7), and EPA (20:53) (obtained from Nu-Check, USA). Tested concentrations were calculated based on the amount of active compound in the FA profile of ethyl esters according to the GC-results.

### In Vivo Treatment Trials

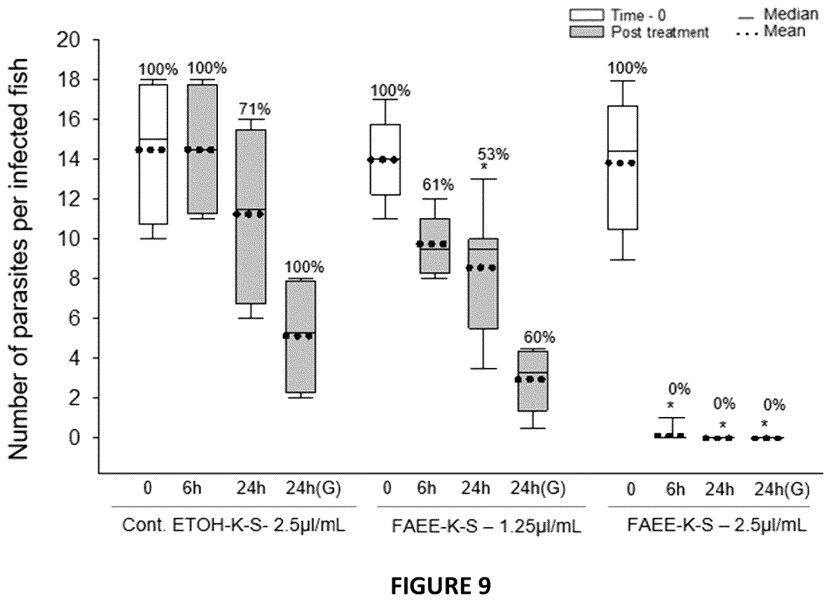

In vivo trials were preceded by a toxicity test to determine the concentrations of the residue extract, FFAs, and FAEEs that do not cause toxicity to fish when applied by immersion (Canadian Council on Animal Care 1998; OECD 2019). A total of 10 fish were placed in 1-L glass beakers (2-3 per each beaker) containing 800 mL of dechlorinated tap water, provided with gentle aeration. Residue ethanolic extract and FFA and FAEE preparations were added at 1.25, 2.5, and 5 μL mL⁻¹, respectively. Fish were observed continuously for 1 h, and then at 1.5, 3, 6, 12, and 24 h for signs of stress, such as abnormal erratic swimming, lethargy, or both. Based on the toxicity test, a safe concentration of 2.5 μL mL⁻¹ was selected as the highest safe level for further in vivo trials, with three replicates for each treatment. Fish exposed to the residue extract and preparations, as part of the toxicity test, were used at the end of the 24 h of exposure. Fish were anaesthetized, and then, the body and the gills were separately fixed in 4% neutral buffered formalin (NBF) and processed for histology. Slides were stained with H&E (hematoxylin and eosin) and observed under a stereomicroscope (Axioskop, Carl Zeiss AG, Germany). Results revealed no toxic effect of the residue extract and preparations on the fish at the tested concentrations (FIG. 7).

An in vivo antiparasitic analysis was performed with fish that were confirmed to be infected. For this, fish were lightly anesthetized in a Petri dish, and the tail fin was observed under a stereomicroscope for the presence of parasites (at least three per fish). The number of parasites per each of the selected fish was recorded and considered as the time 0 level. As described above, 1-3 fish were placed in a 1-L beaker, and the residue extract and preparations were added along with 1% Kolliphor EL and sonication. A total of 10 fish for each treatment were analyzed. Ethanol with 1% Kolliphor EL and sonication and STW were used as negative controls. After 24 h, individual fish were anesthetized and observed under a stereomicroscope, and the number of parasites on their tail fins was recorded.

Revealing the Active Compounds in P. tricornutum Residue Extract and Preparations

GC-FID and lipid separation procedures were performed on the P. tricornutum residue extract and preparations. An in-situ analysis was then performed with the FAEEs that showed the highest proportion in the fatty acid profile in the GC-FID (from the FAEE residue preparation). This preparation was selected for the in vivo activity analysis, as the activity of FFAs from the extract, which was previously described (Kim et al., 2021).

### Lipid Separation

Residue extract and the FFA and FAEE preparations were separated by thin-layer chromatography (TLC), as described by Kim et al., (2021). For the analysis, a total of 150 μL of the extract and the preparations were evaporated under a nitrogen gas stream, resuspended in 40 μL of chloroform, and then resolved on a TLC plate (Silica Gel 60, 10×10 cm, 0.25 mm thickness, Merck, Germany) in one dimension, using a solvent system of hexane: diethyl ether: acetic acid (60:40:1, v/v). A standard neutral lipid mixture, composed of the FFA and TAG, was run in parallel as a reference. Plates were briefly sprayed with iodine vapor, and lipid fractions were scraped off the TLC plate, extracted with chloroform:methanol (2:1, v/v), concentrated, and then analyzed for antiparasitic effect in situ (section 2.6.1) and FA composition by gas chromatography coupled with a flame ionization detector (GC-FID).

### FA Composition Analysis by GC FID

The FA composition of the residue powder and other preparations was determined following a direct transmethylation procedure as described previously by (Kim et al., 2021). The analysis was conducted with 7.5 mg of residue powder; all other preparations were normalized to the same amount of initial residue (7.5 mg, equivalent to 150 μL of the residue extract). Ethanol extracts and hexane fractions of FFA and FAEE were evaporated to dryness under an N₂ gas flow prior to the analysis by GC-FID as described previously (Kim et al., 2021).

### Histopathological Analysis

Fish were sampled from each treatment at the end of the in vivo analysis for histopathological analysis. Anesthetized fish were dissected, and the gills were separately fixed in 4% neutral buffered formalin (NBF) and processed for histology. Slides were stained with H&E (hematoxylin and eosin) and observed under a stereomicroscope (Axioskop, Carl Zeiss AG, Germany). Results revealed no toxic effect of the extract or preparations on the fish at the tested concentrations (FIG. 7).

### Statistical Analysis

Statistical analyses were performed using SigmaPlot software version 13.0 (Systat Software Inc., USA). The in vivo results were analyzed by a one-way ANOVA to compare parasite abundance between different treatment groups, and by a paired t-test to compare parasite abundance before and after treatment within a treatment group. Differences were considered statistically significant at p≤0.05.

### Toxicity Test of Control Treatments

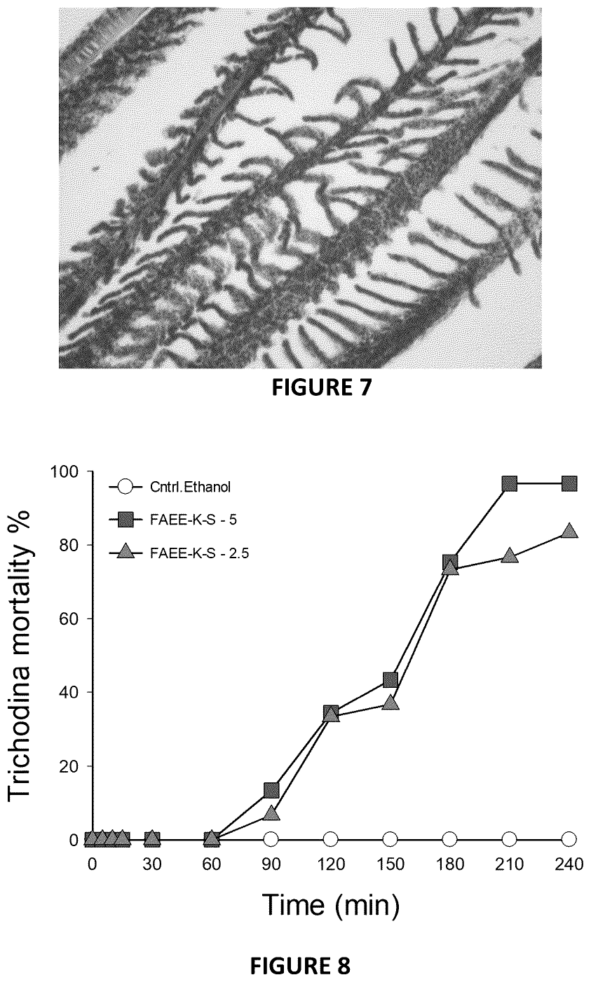

The effects of ethanol, the solvent used for extraction, and the detergent Kolliphor EL (K), in addition to the various manipulations done for extract and fraction preparations, were tested for their antiparasitic effects against G. turnbulli (FIG. 6). The concentrations at which no evident antiparasitic activity occurred was determined as follows: ethanol and ethanol with added 1% (w/v) Kolliphor EL (Ethanol-K) showed no activity against the parasites at a concentration of 10 μL mL⁻¹, whereas Ethanol-K followed by sonication (Ethanol-K-S) showed no activity against the parasites at 5 μL mL⁻¹. Application of the saponification procedure to ethanol and the addition of Kolliphor (1%) prior to analysis (Ethanol (sp)-K) showed no toxicity to the parasites at 10 μl/ml. Application of the saponification procedure to Ethanol-K (Ethanol-K (sp)) produced a solution that showed no activity against the parasites only at a concentration of 2.5 μL mL⁻¹. Based on the above screening results, the concentrations for examining the algal residue extract and fractions were selected as those that showed no effect of the solvent used.

### Experimental Fish

Barramundi fingerlings with heavy infection of Trichodina sp. were obtained from a local aquaculture farm (Aquatech, Ramat Negev, Israel). The infection was confirmed on the fish by examining wet mounts that were collected from skin mucus and gill samples. The parasite was also observed in the water in which the fish were delivered. The infection level was very high (30-40 parasites per field of view in the wet mount samples). Thirty infected fish were placed in 100 L tanks with biological filters and aeration to maintain the infection. Fish were fed once a day with commercial fish food.

### In-Situ Antiparasitic Effect

Infected fish (as confirmed by observing their swimming behavior) were anesthetized with clove oil, followed by euthanization (brain pithing). Gill arches from both sides of the fish were collected and placed in a petri dish with tank water in which the fish were maintained. Using watchmaker's forceps gills were lightly shaken to release the parasite into the water. Then the parasites along with the water were counted and diluted with water to get approximate of 50 per well and then they were transferred to 24 well plate. Preparation of FAEE extract of P. tricornutum residue was added to each well at concentrations of 2.5 and 5 μL mL⁻¹ and the Trichodina were observed every 30 min for motility under an inverted microscope. Non-viable Trichodina were considered dead and percent parasite mortality was calculated. For control, solvent only (ethanol) was used.

### In Vivo Antiparasitic Effect

Three aquaria with 3 L water each was set up with aeration. Two fish with the infection were placed in each aquarium. The trial was conducted by addition of FAEE extract of P. tricornutum residue at concentrations of 1.25 and 2.5 μl/ml, and water was used as control. Parasites were counted at times 0, 6, and 24 h from the skin surface of lightly anesthetized fish. Wet mounts of fish skin, collected from about 4 cm2 of skin surface were collected with a cover slip and observed under a light microscope the coverslip and parasites were counted. After 24 h fish were deeply anesthetized the 3^{rd} gill arches from both sides were collected and examined for presence of parasites under a light microscope.

### Example 1

### Phaeodactylum tricornutum Residue Extract and Preparations as Treatment

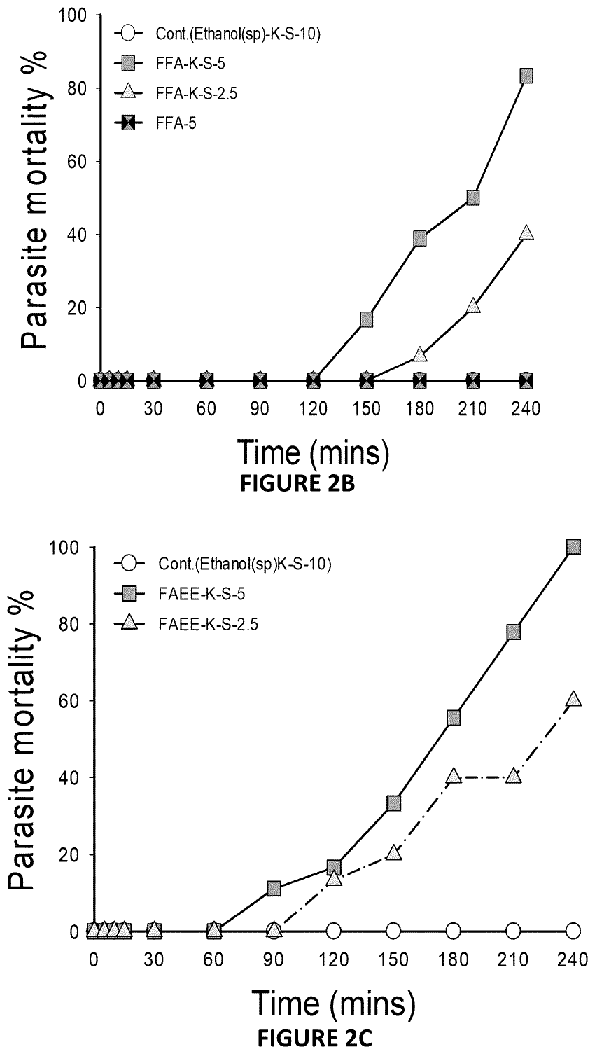

The residue extract (RE) prepared from the P. tricornutum residual material (2,000 μl/100 mg) showed no antiparasitic activity up to a concentration of 10 μL mL⁻¹. The addition of the detergent, 1% Kolliphor EL (w/v) (RE-K), during extraction induced 100% parasite mortality at the same concentration of 10 μL mL⁻¹ within 240 min, whereas no antiparasitic activity was detected at the lower concentration of 5 μL mL⁻¹ (FIG. 2A). The differences in the antiparasitic activity of RE with and without Kolliphor EL suggest that this detergent and sonication help solubilize and homogenize the hydrophobic compounds present in the residue extract. However, considering the starting amount of residue, the application rate (10 μL mL⁻¹) was relatively high. Further experiments were then performed, aiming to increase the efficacy and reduce the application dose to less than 5 μL mL⁻¹.

Fatty acids were quantified by GC-FID in the residue powder (Table 1). The inventors next applied saponification to prepare FFAs from the fatty acids associated with the residue extract. The inventors have previously shown that FFAs, which can be released from the glycerolipids of P. tricornutum, display anti-monogenean activity (Kim et al., 2021). The FFA fraction that was dissolved in ethanol and applied in the assay at 5 μL mL⁻¹ was not active against the parasite. However, with the addition of 1% Kolliphor EL in the stock solution and after sonication, it induced 40% and 85% parasite mortality at application rates of 2.5 and 5 μL mL⁻¹, respectively, within 240 min (FIG. 2B). The antiparasitic activity of the FFA fraction prepared from the RE was higher than the activity of the original RE-K applied at the same dose of 5 μL mL⁻¹ (FIG. 2A).

To simplify the procedure and eliminate the need for prior extraction and saponification, fatty acid ethyl esters (FAEEs) were produced by direct transmethylation of the residue. FAEEs solubilized in ethanol, with the addition of 1% Kolliphor EL and sonication, induced 40% and 100% parasite mortality within 240 min at concentrations of 2.5 and 5 μL mL⁻¹, respectively (FIG. 2C). This marked an increase in the activity as compared to the residue extract and the FFA, reducing the minimum activity level to 2.5 μL mL⁻¹, a concentration at which the other preparations displayed no activity.

### Example 2

### Antiparasitic Activity In Vivo

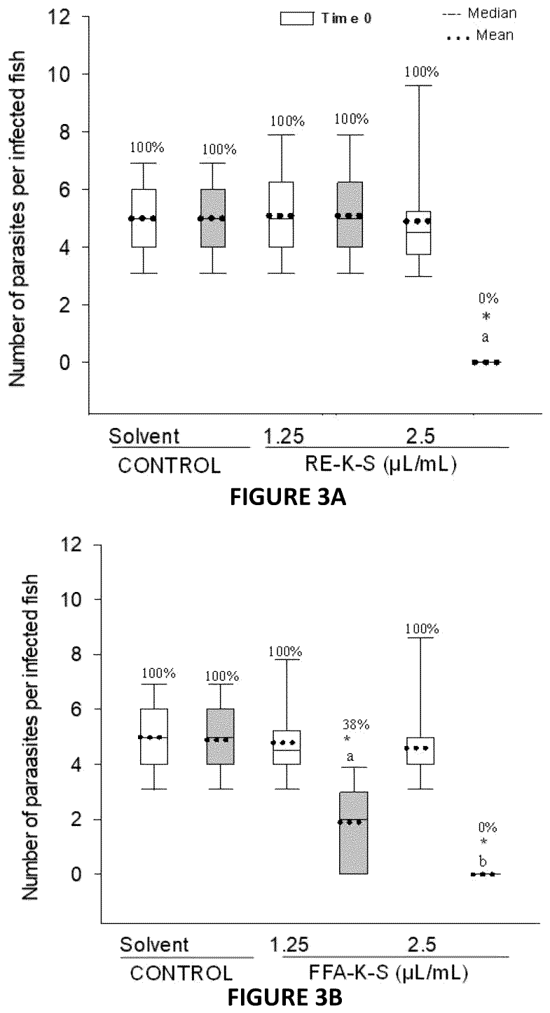

The in vivo effect of the residue extract and preparations was tested with added 1% Kolliphor EL and sonication. Based on the preliminary toxicity test, infected fish were exposed to residue extract and preparations at concentrations of 1.25 and 2.5 μL mL⁻¹ for 24 h. Residue extract at 2.5 μL mL⁻¹, applied for 24 h, eliminated the infection, whereas RE at 1.25 μL mL⁻¹ and the negative control resulted in no change in infection after the treatment (FIG. 3A). The FFA at 1.25 μL mL⁻¹ reduced the infection prevalence from 100% to 38% after 24 h, and application at 2.5 μL mL⁻¹ eliminated the infection. The negative control showed no change in infection after the treatment (FIG. 3B). The results show that the FFA preparation was effective at a lower concentration than the residue extract (FIG. 3A).

The FAEE at 1.25 μL mL⁻¹ reduced the infection prevalence from 100% to 28% after 24 h, and application at 2.5 μL mL⁻¹ eliminated the infection completely. The negative control showed no change in infection after the treatment (FIG. 3C). The FAEE preparation was more effective than the FFA at the same concentration of 1.5 μL mL⁻¹ (FIG. 2B).

### Example 3

### Revealing the Active Anti-G. turnbulli Compounds in P. tricornutum Residue Extract and Preparations

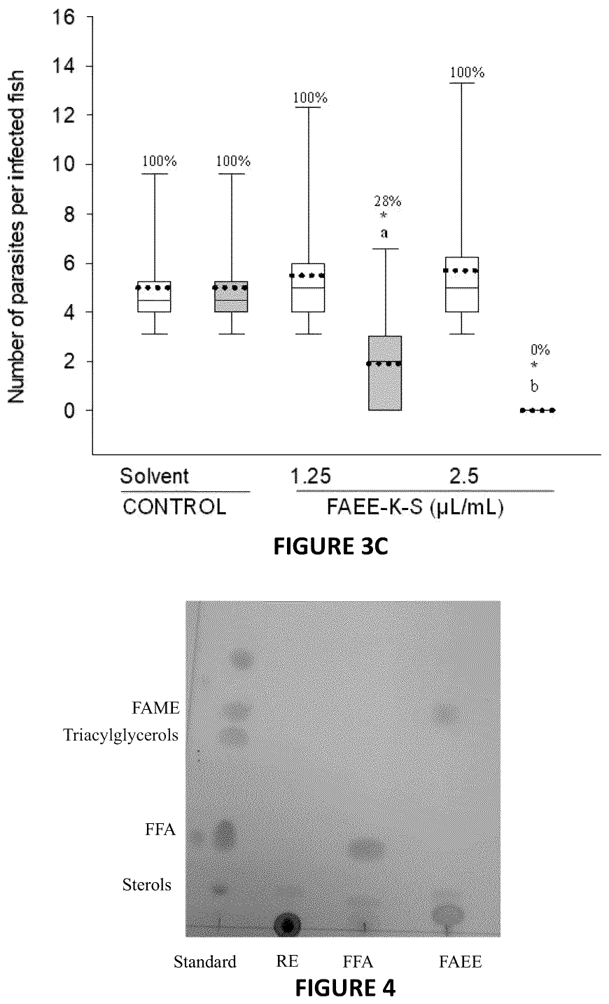

The fatty acid compositions of P. tricornutum residue, the residue extract (RE), and the preparations (FFA & FAEE) were analyzed by GC-FID, and the total amount of FAs was calculated as μg mg⁻¹. The FAEE preparation contained a total of 46.1 μg mg⁻¹ of FAs, which was the highest level, followed by the original residue powder, which contained 39.2 μg mg⁻¹. The FFA preparation and the RE contained ca. 21 μg mg⁻¹. The FA composition in all P. tricornutum samples comprised four major FAs: myristic acid (14:0), palmitic acid (16:0), palmitoleic acid (16:1n7), and EPA (20:5n3). The RE and different fractions were resolved by TLC to test whether the RE contained FFAs and to confirm that the conversion of lipid-bound fatty acids to FFAs and FAEEs was complete (FIG. 4). The RE did not contain FAs in their free form, whereas residual polar lipids and pigments were visible at the front of the TLC plate.

TABLE 1
Major fatty acid (FA) composition (including FAs present at >1% of total FAs)
of P. tricornutum residue, ethanolic residue extract, and FFA and FAEE preparations:
analysis was carried out by GC-FID (an average of three replicates).

| | Percentage (%) | | | | Amount (μg mg⁻¹) * | | | |
|---|---|---|---|---|---|---|---|---|
| | | | | FAEE | | | | FAEE |
| Fatty acids | Residue | RE | FFA | (R) | Residue | RE | FFA | (R) |
| 14:0 | 11.2 | 4.5 | 6.7 | 11.0 | 4.7 | 1.0 | 1.6 | 5.6 |
| 14:1 | — | 1.0 | — | 0.5 | — | 0.2 | — | 0.3 |
| 16:0 | 13.7 | 7.1 | 11.2 | 14.2 | 5.7 | 1.6 | 2.7 | 7.3 |
| 16:1 | 26.2 | 19.8 | 21.4 | 24.0 | 11.0 | 4.5 | 5.1 | 12.3 |
| 16:2 | 7.1 | 7.9 | 7.3 | 6.2 | 3.0 | 1.8 | 1.7 | 3.2 |
| 16:3 | 1.2 | 2.2 | 1.5 | 1.0 | 0.5 | 0.5 | 0.4 | 0.5 |
| 16:4 | 1.6 | 1.7 | 1.3 | 0.9 | 0.7 | 0.4 | 0.3 | 0.4 |
| 18.0 | 0.4 | 0.7 | 7.0 | 3.0 | 0.2 | 0.4 | 1.7 | 1.5 |
| 18:1n9 | 2.6 | 4.2 | 2.4 | 0.9 | 1.1 | 0.9 | 0.6 | 0.5 |
| 18:2 | 1.9 | 4.0 | 5.1 | 2.2 | 0.8 | 0.9 | 1.2 | 1.1 |
| 18:3n3 | 0.3 | 0.7 | 0.6 | 0.8 | 0.1 | 0.2 | 0.2 | 0.4 |
| 18:3n6 | 0.9 | 1.7 | 1.5 | 0.9 | 0.4 | 0.4 | 0.4 | 0.5 |
| 18:4 | 0.3 | 3.5 | 0.5 | 0.4 | 0.1 | 0.8 | 0.1 | 0.2 |
| 20:3n6 | 0.3 | 2.9 | — | 0.5 | 0.1 | 0.7 | — | 0.3 |
| 20:4n6 | 1.6 | 1.8 | 1.5 | 1.4 | 0.7 | 0.4 | 0.4 | 0.7 |
| 20:5n3 (EPA) | 22.4 | 24.3 | 21.8 | 20.4 | 9.4 | 5.5 | 5.2 | 10.5 |
| 22:0 | 1.7 | 2.1 | — | 0.3 | 0.7 | 0.5 | — | 0.2 |
| 22:1 | — | 1.7 | 0.9 | 1.1 | — | 0.4 | 0.2 | 0.6 |
| Total fatty acids | | | | | 39.2 | 21.1 | 21.8 | 46.1 |

* mg of P. tricornutum biomass as dry weight

### Example 4

### In Situ Antiparasitic Activity of Pure (Commercial) Active Compounds

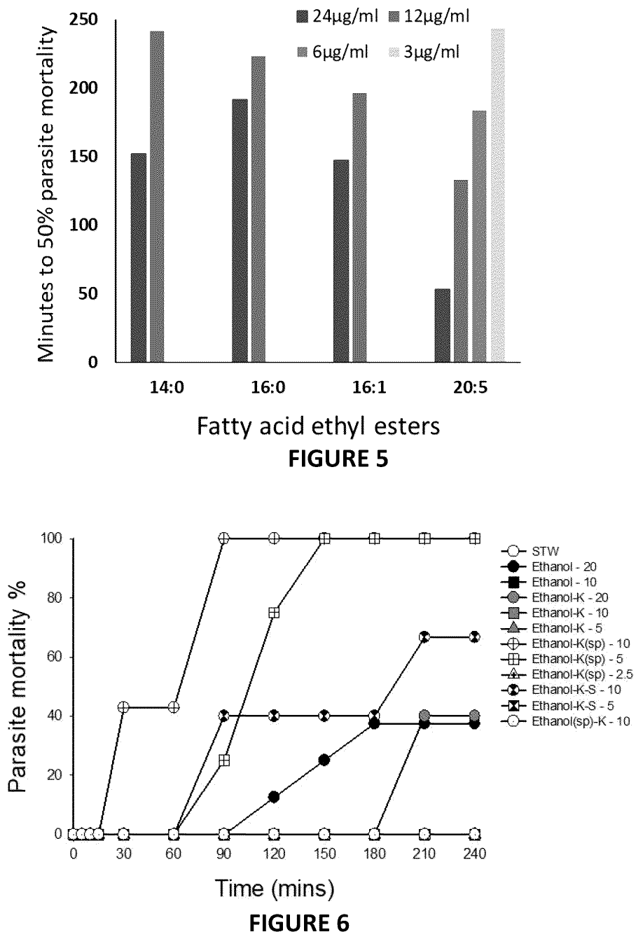

The antiparasitic activity of the major FAs found in the residue, its ethanolic extract, and the preparations was tested in their ethyl ester form (obtained as commercial material). Results revealed the activity of all four FAEEs: of myristic acid (14:0), palmitic acid (16:0), palmitoleic acid (16:1n7), and EPA (20:5n3). Of these, EPA-EE was the most active, inducing 50% parasite mortality in the shortest time for the higher concentrations tested, 12 and 24 μL mL⁻¹, and inducing parasite mortality at 3 and 6 μL mL⁻¹, concentrations at which the other FAEEs no longer affected the parasite. Moreover, EPA-EE showed a dose-dependent effect (FIG. 5).

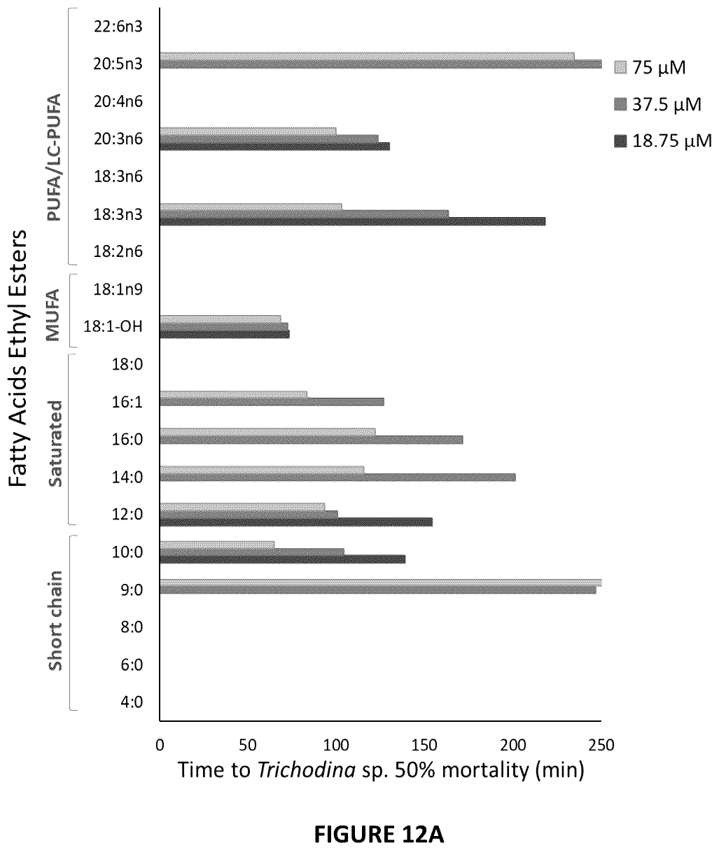

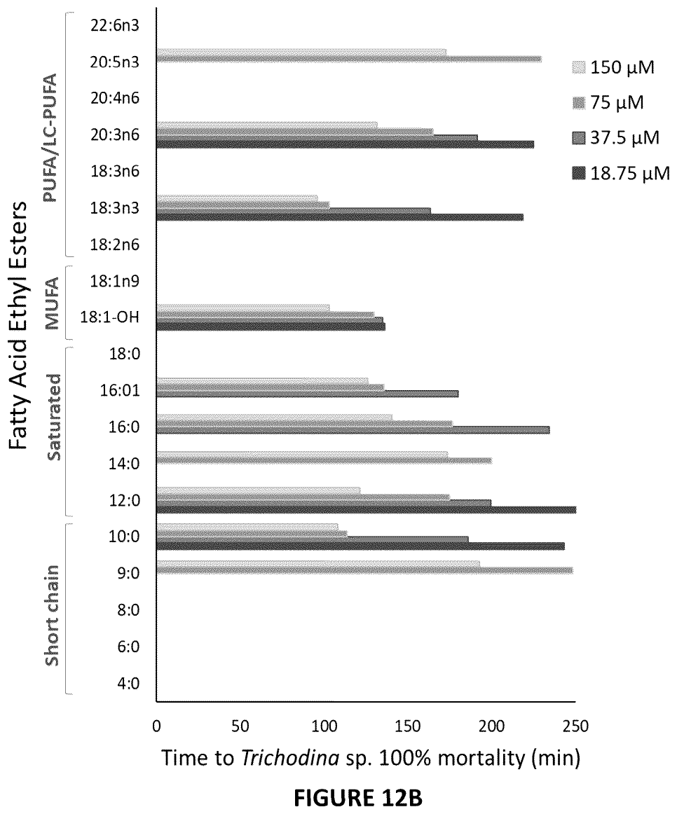

Further, the inventors examined antiparasitic activity of a broader scope of FFAEs in-situ. Specifically, short chain (4:0, 6:0, 8:0; 9:0, and 10:0), saturated (12:0, 14:0, 16:0, and 18:0), monounsaturated fatty acid (MUFA; 18:1-OH, and 18:1n9), and polyunsaturated fatty acid (PUFA)/long chain PUFA (LC-PUFA; 18:2n6, 18:3n3, 18:3n6, 20:3n6, 20:4n6, 20:5n3, and 22:6n3) fatty acids were tested for their antiparasitic activity on Gyrodactylus and Trichodina (FIGS. 11-12, respectively).

The results show that any one of: short chain (6:0, 8:0; 9:0, and 10:0), saturated (12:0), MUFA (18:1-OH), and PUFA/LC-PUFA (18:2n6, 18:3n3, 18:3n6, and 20:5n3), induced an antiparasitic activity in the context of Gyrodactylus in situ, providing at least 50% mortality when applied at a concentration ranging between 18.75 μM and 75 μM (FIG. 11A). Further, when the concentration of the tested FFAEs was increased to 150 μM or 300 μM, in addition to the species which were found to exert antiparasitic activity in FIG. 11A, the short chain (4:0), saturated (14:0, and 16:0), and PUFA/LC-PUFA (20:3n6) fatty acids were also found to induce an antiparasitic activity in the context of Gyrodactylus in situ, providing at least 100% (FIG. 11B).

The results show that any one of: short chain (9:0), saturated (12:0, 14:0, and 16:0), MUFA (18:1-OH), and PUFA/LC-PUFA (18:3n3, 20:3n6, and 20:5n3), induced an antiparasitic activity in the context of Trichodina in situ, providing at least 50% or 100% mortality when applied at a concentration ranging between 18.75 μM and 75 μM (FIG. 12A), or between 18.75 μM and 150 UM (FIG. 12B), respectively.

Further, the short chain (9:0, at a concentration ranging from 18.75 μM and 37.5 μM), saturated (10:0, at a concentration ranging from 18.75 μM and 37.5 μM; 12:0, at a concentration ranging from 9.375 μM and 37.5 M; 14:0, at a concentration ranging from 7.3 μM and 14.5 M; and 16:0, at a concentration ranging from 7.3 μM and 14.5 μM), and PUFA/LC-PUFA (18:2n6, at a concentration ranging from 7.3 μM and 14.5 μM) fatty acids; 18:3n3, at a concentration ranging from 7.3 μM and 14.5 μM; 18:3n6, at a concentration ranging from 7.3 μM and 14.5 M; and 20:5n3, at a concentration ranging from 7.3 μM and 14.5 μM) fatty acids, were found to be non-toxic to fish over a period of at least 48 h.

### Discussion

Microalgae have a range of industrial applications as primary producers of many valuable compounds, such as pigments, lipids, proteins, polysaccharides, vitamins, and minerals. These compounds display potent biological activities, including antioxidant, anticoagulant, anti-inflammatory, antimicrobial, and antitumor effects.

In the production of high value compounds, industrial companies produce large amounts of algal residue after extracting the desired bioactive compound. These residues, which need to be cleared from the production site, contain a range of compounds that can be further utilized. The residue's composition depends on the original target material for production, the type of algae, and the fatty acid profile.

Phaeodactylum tricornutum is a marine diatom, which grows in both brackish and marine waters. P. tricornutum contains four main FAs as the primary components of membrane polar lipids: myristic acid (14:0), palmitic acid (16:0), palmitoleic acid (16:1n7), and EPA (20:5n3). Desbois et al. (2009) found that fatty acids from P. tricornutum display potent antibacterial activity against a range of bacterial pathogens, including the very dangerous human pathogen MRSA. They have identified three unsaturated fatty acids involved in this antibacterial activity: the polyunsaturated fatty acid EPA (20:53), the monounsaturated palmitoleic acid (16:1n7), and the polyunsaturated hexadecatrienoic acid (HTA) (Desbios et al., 2008, 2009).

Of the main fatty acids present in the P. tricornutum ethanolic extract, 16:1n7 and EPA had toxic effects on two major bacterial pathogens of fish: the Gram-positive S. iniae and the Gram-negative V. harveyi. EPA was also reported to be effective against the marine bacterium Listonella anguilarum, which affects aquatic organisms. Antibacterial activity was demonstrated in additional microalgal species, including Chlorella pyrenoidosa, Scenedesmus quadricauda, and Dunaliella primolecta. In these species, the antibacterial activity was related to several bioactive compounds, including pigments, such as phycobiliproteins and chlorophyll derivatives; however, in most cases, the activity was associated with free fatty acids.

Fatty acids' antiparasitic effects against protozoan parasites have previously been demonstrated. These parasites, including Plasmodium falciparum and Trichomonas vaginalis, are characterized by having no cell wall. A range of action modes for the antiparasitic activity have been proposed. The exposure of Plasmodium falciparum, malaria's causative agent, to FA fractions obtained from the marine sponge Agelas oroides resulted in the inhibition of endogenous FA production and parasite death. LC-PUFA's antiparasitic effect against P. falciparum was related to stimulation of the host's immune system, resulting in effective antiparasitic activity against the intracellular parasite.

The surface layer of Gyrodactylus is a syncytial epidermis. The lack of a cell wall in the G. turnbulli epidermis may explain the detrimental effect that FAs have on the parasite following exposure in situ and in vivo. The inventors observed that the parasite's shape was altered in the in vivo assay following exposure to the active fractions, and the dead and detached parasite appeared to be swollen. This may suggest that the site of activity against G. turnbulli is the parasite's integument, but further research is required to confirm this assumption and reveal the actual mode of action.

The commercial microalgal producer Algatech Ltd. (Ketorah, Israel) grows P. tricornutum for fucoxanthin, which is used for preparing commercial health products for human consumption. The residue of P. tricornutum after fucoxanthin extraction was used in this study to produce a preparation for treating G. turnbulli in guppies. This was based on a previous study that revealed the efficacy of P. tricornutum extract application as an anti-monogenean disease treatment and identified the active compounds as free FAs. In the current study, the aim was to recover FAs in various preparations from the residue and to apply manipulations to enhance the activity, safety, stability, or any combination thereof, of the antiparasitic preparation.

Comparing and testing different extraction methods and manipulations revealed that the application of transmethylation, which liberated the FAs from the original residue material in the form of fatty acid ethyl esters (FAEEs), was the most effective in terms of obtaining the largest amount of FAs from the residue and providing maximal activity per gram of the original biomass (P. tricornutum residue). The active FAEEs were myristic acid (14:0), palmitic acid (16:0), palmitoleic acid (16:1n7), and EPA (20:5n3), tested in vivo as pure compounds. As with the FFAs (Kim et al. 2021), EPA-EE was the most active.

Although not directly compared in the current study, it appears that the FAs in their ethyl ester form (FAEE) are less active than in their free form (FFA). When examining the amount of FAs in the extracts and preparations, it was evident that the FAEE preparation from the residue had approximately double the amount of FAs (about 46 μg mL⁻¹) as compared to the RE and the FFA preparation (about 21 μg mL⁻¹) prepared from the extract, due to the better recovery of active material. Yet the increase in activity was not as substantial in the exposure to 5 μg mL⁻¹ for 240 min, with FAEE mortality reaching 100%, while with FFA, it reached 90%. Indeed, FAEEs were reported to be less prone to oxidation than FFAs; thus, the preparation would have a longer shelf life and is likely to be stable when kept at chilling temperatures. This does require further investigation for the algal preparation.

Overall, obtaining and using the FAs from the algal material as FAEEs possesses a range of advantages. Its production from the algal material is a one-step process (direct transethylation), which reduces the barriers and costs associated with the extraction procedure. FAEEs are less toxic and more stable than FFAs. There is the potential for further use of the residual medium obtained after the transmethylation procedure, as it is rich in nutrients, including glycerol, amino acids, and sugars originating from the algal material. Neutralizing the acidity of this medium with a base (such as ammonium-hydroxide), for example, can produce a bio-fertilizer and bio-stimulant for plant or algal production.

Microalgae are a rich untapped resource for bioactive molecules, as commercial algae production, in many cases, produces residues that can be used for various biorefinery products. Screening for such compounds, which can be active against various pathogenic microorganisms, will increase the production of eco-friendly treatments for aquacultural diseases and will also contribute to the sustainable utilization of algal byproducts and waste. Moreover, the processes of algal production and extraction entail high implementation and operational costs. In addition, energetic requirements represent a significant component of the process's sustainability. According to Grima et al., 2003, extraction can account for up to 60% of overall expenditures. The utilization of microalgal waste and byproducts for the production of additional commercial items can increase the profitability of microalgal production.

The current study provided exemplary scientific evidence in a specific monogenean fish parasite, G. turnbulli, and the protozoan parasite Trichodina sp. nonetheless, the inventors suggest that the extract from P. tricornutum biomass and residue and specifically FAAE, could be used against additional monogenean species, as well as other parasites (Trichodina, Tetrahymena) negatively affecting aquatic organisms.

### Example 5

### Antiparasitic Effect of Fatty Acids Ethyl-Esters (FAEE(R)) of Algal Residue Powder

The anti-parasitic activity of FAEE of the algal residue was tested against a common protozoan parasite, Trichodina sp., which was obtained from an outbreak that occurred at a commercial food fish farm.

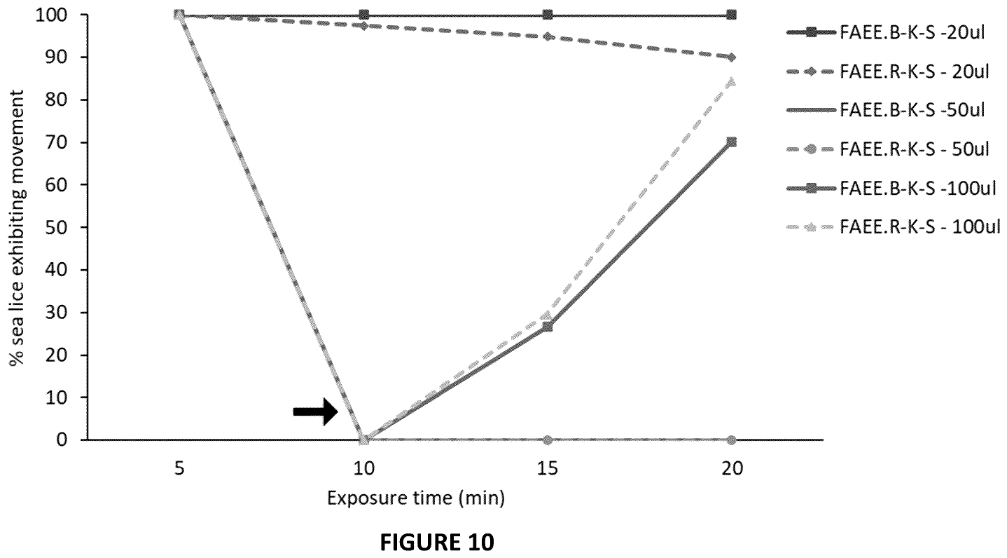

In vitro analysis revealed that FAEE fraction with added 1% Kolliphor and sonication induced 83% and 96% parasite mortality at concentrations of 2.5 and 5 μl/mL respectively, within 240 min (FIG. 8).

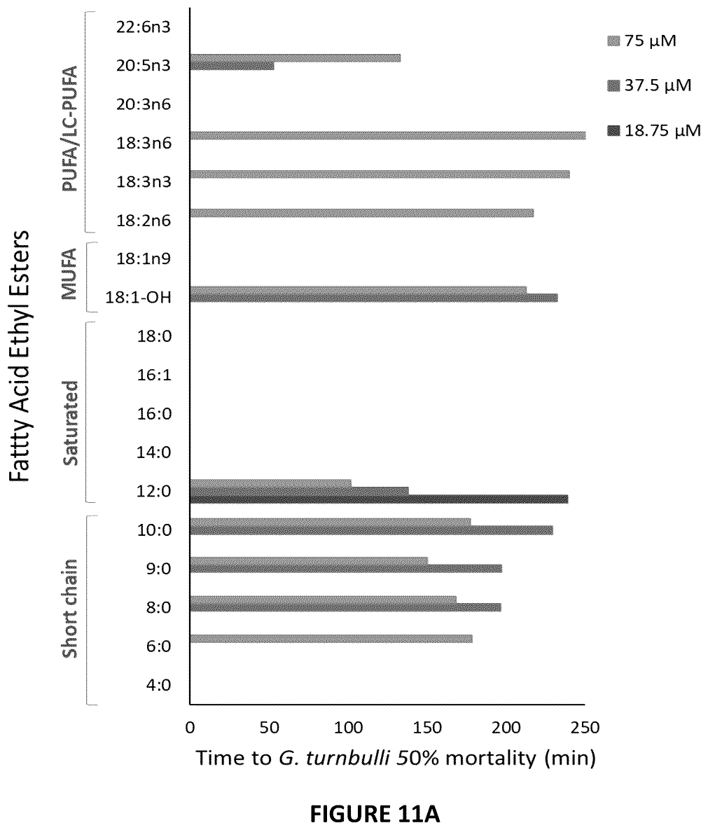

In vivo analysis revealed complete clearance of the parasites from the skin and gills of the fish following a bath treatment at a concentration of μl/mL (Table 2 and FIG. 9).

TABLE 2
In vivo treatment with FAEE of infected barramundi

| Treatment | | | | | Parasites on | |
|---|---|---|---|---|---|---|
| (FAEE | | Parasites on the skin¹ | | | the gills (24 h) | |
| μl/mL) | Fish # | Time 0 | Time 6 h | Time 24 h | Gill 1 | Gill 2 |
| 1.25 | 1 | 32 | 12 | 3.5 | 1 | 0 |
| | 2 | 14 | 9 | 4.5 | 0 | 2 |
| 2.5 | 1 | 17 | 0 | 0 | 0 | 0 |
| | 2 | 20 | 0 | 0 | 0 | 0 |
| Control | 1 | 23 | 18 | 9 | 2 | 2 |
| (water) | 2 | 17 | 23 | 6 | 0 | 6 |

¹Examination of 2 skin samples, each collected from 4 cm² area. The average is reported

### Example 6

### In Situ Anti-Lice Activity

The inventors have further tested the antiparasitic effect of the FAEE disclosed herein on sea lice in-situ. The FAEE were prepared from whole biomass and residue of algae P. tricornutum, as described herein. In the analyses, a food grade detergent, Kolliphor 1% (K) and sonication (S) were added to FAEE prior to analysis.

The analyses were carried out in-situ, with the chalimus II (parasitic stage of sea lice) placed in cell strainer immersed in petri dish containing FAEE solution in sea water. In the in-situ trials, sea lice were observed for mortality for a time period of 10 min, and then transferred to clean seawater to check for their recovery. Recovery followed for 20 mins.

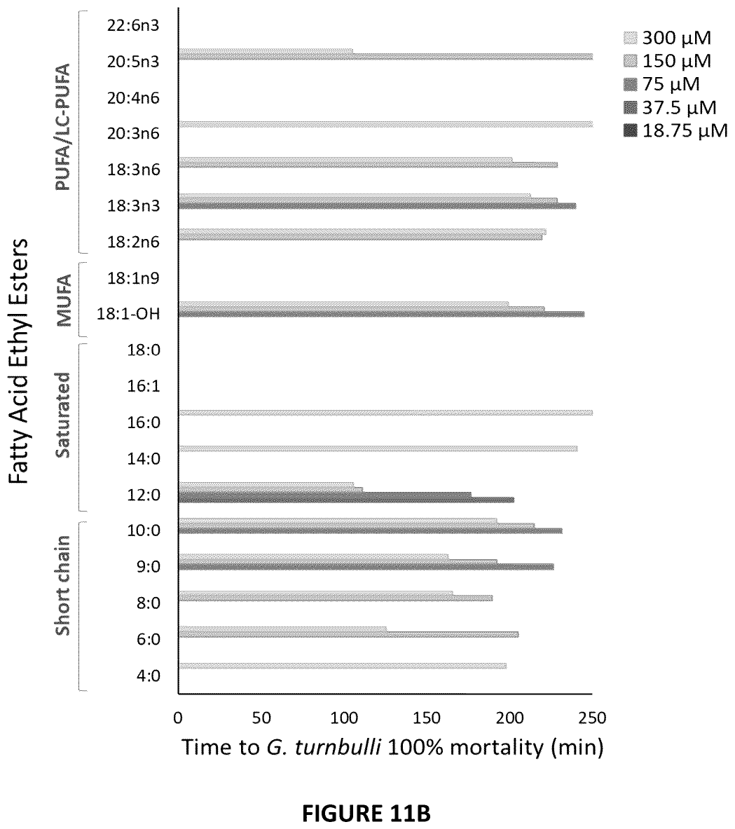

The results show immobilization of the parasites was achieved in treatment with FAEEs of residue from P. tricornutum at any of the tested concentrations (20, 50, and 100 μL mL⁻¹; FIG. 10).

At concentration of ≥50 μL/mL full immobilization was achieved within 10 min.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method for preventing or treating a pathogenic infection in an aquatic organism in need thereof, the method comprising administering to said aquatic organism a therapeutically effective amount of an esterified fatty acid, comprising a fatty acid chain of 4 to 24 carbon atoms.

2. The method of claim 1, wherein said esterified fatty acid is ethyl ester, methyl ester, butyl ester, propyl ester, or a combination thereof.

3. The method of claim 1, wherein: (i) said esterified fatty acid comprises a plurality of types of esterified fatty acids, wherein each of said esterified fatty acids comprises a fatty acid chain of 4 to 24 carbon atoms; (ii) said esterified fatty acid comprises an esterified fatty acid being selected from the group consisting of: eicosapentaenoic acid (EPA), myristic acid, palmitic acid, palmitoleic acid, and any combination thereof; (iii) said esterified fatty acid comprises an esterified EPA; (iv) said fatty acid chain is of 9 to 22 carbon atoms; or (v) any combination of (i) to (iv).

4.-6. (canceled)

7. The method of claim 1, wherein said fatty acid is a saturated fatty acid or an unsaturated fatty acid, with 1 to 6 double bonds.

8. (canceled)

9. The method of claim 1, wherein said composition further comprises a detergent, and optionally wherein said detergent is present in said composition in an amount of 0.1-5% by weight of said composition.

10. (canceled)

11. The method of claim 1, wherein: (i) said pathogenic infection excludes bacterial infection; (ii) said administering comprises contacting a body of water comprising said aquatic organism with said esterified fatty acid, and optionally wherein said method comprises contacting said body of water with an amount of 0.1 to 500 μg of said esterified fatty acid per ml of water of said body of water, contacting said body of water with an amount of 1 to 50 μg of said esterified fatty acid per ml of water of said body of water, or both; or (iii) both (i) and (ii).

12.-14. (canceled)

15. The method of claim 1, wherein: (i) said aquatic organism is selected from the group consisting of: fish, crustacean, mollusk, and any combination thereof; (ii) said pathogenic infection comprises any one of a monogenean infection, a protozoan infection, or both, and optionally wherein monogenean infection is induced by Gyrodactylus turnbulli, said protozoan infection is induced by a protozoan belonging to any one of the genera Trichodina and Tetrahymena, or both; or (iii) both (i) and (ii).

16.-18. (canceled)

19. A composition comprising an esterified fatty acid, comprising a fatty acid chain of 4 to 24 carbon atoms, and an agriculturally or aquaculturally acceptable carrier, for prevention, treatment, or both, of a pathogenic infection in an aquatic organism in need thereof.

20. The composition of claim 19, further comprising a detergent, and optionally wherein said detergent is present in said composition in an amount of 0.1-5% by weight of said composition, said detergent comprises Kolliphor EL, or both.

21.-22. (canceled)

23. The composition of claim 19, comprising an extract of a microalga, or any fraction thereof, and optionally wherein said microalga is selected from the group consisting of: Phaeodactylum tricornutum, Odontella aurita, Nitzschia laevis, Nannochloropsis oceanica, Nannochloropsis limnetica, Nannochloropsis oculata, Microchloropsis gaditana, Microchloropsis salina, Monodopsis subterranean, Tribonema sp, and any combination thereof.

24. (canceled)

25. The composition of claim 19, wherein said esterified fatty acid is ethyl ester, methyl ester, butyl ester, propyl ester, or a combination thereof.

26. The composition of claim 19, wherein said esterified fatty acid comprises a plurality of types of esterified fatty acids, wherein each of said esterified fatty acids comprises a fatty acid chain of 4 to 24 carbon atoms.

27. The composition of claim 19, wherein said esterified fatty acid comprises an esterified fatty acid being selected from the group consisting of: eicosapentaenoic acid (EPA), myristic acid, palmitic acid, palmitoleic acid, and any combination thereof.

28. The composition of claim 19, wherein said esterified fatty acid comprises an esterified EPA.

29. The composition of claim 19, wherein said fatty acid chain is of 9 to 22 carbon atoms.

30. The composition of claim 19, wherein said pathogenic infection excludes bacterial infection.

31. The composition of claim 19, wherein said pathogenic infection comprises any one of a monogenean infection, a protozoan infection, or both, and optionally wherein monogenean infection is induced by Gyrodactylus turnbulli, said protozoan infection is induced by a protozoan belonging to any one of the genera Trichodina and Tetrahymena, or both.

32.-33. (canceled)

34. The composition of claim 19, being administered or supplemented into a body of water comprising said aquatic organism, such that a concentration of 1 to 50 μg of said esterified fatty acid per ml of water of said body of water is obtained.

35. The composition of claim 19, wherein said aquatic organism is selected from the group consisting of: fish, crustacean, mollusk, and any combination thereof.

36. The composition of claim 19, formulated for administration to a body of water, and optionally wherein said body of water comprises said aquatic organism, said composition is formulated for administration to an aquatic organism while residing, being cultured, or both, in said body of water, or both.

37.-38. (canceled)
